(19) United States
(12) Patent Application Publication
(10) Pub. No.: US 2010/0272638 A1
(43) Pub. Date: Oct. 28, 2010

(54) RADIOLABELLED MICROPARTICLES, PROCESSES FOR THE PREPARATION THEREOF AND THE USE THEREOF

(75) Inventors: Mareke HARTIG, Ingelheim (DE); Michael Trunk, Ingelheim (DE); Thomas Weuthen, Gilching (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: Boehringer Ingelheim International GmbH, Ingelheim (DE)

(21) Appl. No.: 12/706,363

(22) Filed: Feb. 16, 2010

Related U.S. Application Data
(63) Continuation of application No. 10/824,566, filed on Apr. 14, 2004, now abandoned.
(60) Provisional application No. 60/478,295, filed on Jun. 13, 2003.

(30) Foreign Application Priority Data
Apr. 16, 2003 (DE) 10317461.3

(51) Int. Cl. A61K 51/12; A61P 11/00

(52) U.S. Cl. 424/1.37

(57) ABSTRACT

The invention relates to microparticles radioactively labelled with the technetium isotope ^{99m} Tc, a process for the preparation thereof and their use for preparing powdered inhalants—suitable for deposition studies, for example—which contain the labelled microparticles as active substances.

### RELATED APPLICATIONS

Benefit of U.S. Provisional Application Ser. No. 60/478,295, filed on Jun. 13, 2003 is hereby claimed, and which application is incorporated herein in its entirety.

### FIELD OF INVENTION

The invention relates to microparticles radioactively labelled with the technetium isotope ^{99m}Tc, a process for the preparation thereof, and their use for preparing powdered inhalants—suitable for deposition studies, for example—which contain the labelled microparticles as active substances.

The radioactive powdered inhalants prepared by this method do not differ either in their physico-chemical and technical properties or in their medical applications from the corresponding unlabelled/analogous powdered inhalants which are manufactured on an industrial scale as pharmaceutical compositions (commercial products).

### BACKGROUND TO THE INVENTION

In “powdered inhalant” preparations, inhalable powders packed into suitable capsules (inhalettes), for example, are delivered by means of powder inhalers. Alternatively, they may also be administered by inhalation by the use of suitable powdered inhalable aerosols which contain HFA134a, HFA227 or mixtures thereof as the propellant gas, for example.

Normally, powdered inhalants are produced, e.g. in the form of capsules for inhalation, on the basis of the general teaching as described in DE-A-179 22 07. A significant aspect of the administration of an active substance by inhalation is that only particles of a certain aerodynamic size reach the target organ, the lungs. The particle size of these lung-bound particles (inhalable fraction) is in the region of a few microns. Such particles are usually produced by micronisation (e.g. air jet grinding). Apart from the jet grinding process it is also possible to produce a suitably micronised product by alternative methods. It is known from the literature to produce inhalable microparticles by spray drying. Normally, industrially useable formulations which exhibit sufficient dispersibility in medical use (inhalation) are prepared from spray-dried particles of this kind using the method mentioned above (DE-A-179 22 07) [Y.-F. Maa, P.-a. Ngyuyen, J. D. Andya, N. Dasovich, T. D. Sweeny, S. J. Shire, C. C. Hsu, Pharmaceutical Research, 15, No. 5 (1998), 768-775; M. T. Vidgrén, P.-A. Vidgrén, T. P. Paronen, Int. J. Pharmaceutics, 35 (1987), 139-144; R. W. Niven, F. D. Lott, A. Y. Ip, J. M. Cribbs, Pharmaceutical Research, 11, No. 8 (1994), 1101-1109]. In formulations of this kind, a uniform distribution of the pharmaceutical composition in the powder mixture (formulation) is one of the critical factors.

The efficacy and site of activity of a pharmaceutical composition—particularly when inhaled—may be determined using a deposition study in which the active substance is radioactively labelled and then its distribution in the human body is detected by scintigraphy. In order to guarantee a clear, quantitative correlation between the active substance distribution and the radioactive distribution, the labelling process and the formulation must be designed so that the quantitative deposition of the active substance in the lungs corresponds precisely to the quantitative distribution of the radioactivity.

For solution aerosols which are inhaled in the form of ultrafine micro-droplets, the technical prerequisite—assuming a homogeneous active substance distribution—is that the radioactive substance must be homogeneously distributed in the same way in the solution which is to be sprayed. Usually this is achieved by homogeneously dissolving the radioactive substance in the active substance solution [K. P. Steed, L. J. Towse, B. Freund, S. P. Newman; Eur. J. Pharm. Sci. (1997), 5 (2), 55-61].

The theoretical procedure in the radioactive labelling of propellant-driven MDIs differs only slightly from the labelling process for nebuliser formulations: in the suspension formulation consisting of a micronised active substance present in the form of solid particles, and the pressurised propellant gas which is present in liquid phase, the radioactive substance is homogeneously dissolved in the liquid propellant gas medium. During application (atomisation) the propellant gas is nebulised spontaneously in the micro-droplets produced by the spraying operation, which consist of propellant gas (liquid), radioactive substance (dissolved) and micronised active substance (suspended), and the radioactive substance is deposited on the microparticles of active substance. In this procedure, too, a direct quantitative correlation between active substance and radioactive labelling is also guaranteed [S. P. Newman, A. R. Clark, N. Talaee, S. W. Clarke; Thorax (1989), 44, 706-710; R. Pauwels, S. Newman, L. Bergstrom; Eur. Respiratory J. (1997), 10 (9), 2127-2138].

The methods of radioactively labelling active substances described above are not applicable to powdered formulations, if these formulations consist of a powder mixture with at least one pharmacologically inactive carrier material (solid) and at least one micronised active substance (solid). For systems of this kind, in addition to the quantitative correlation between radioactivity and active substance mentioned above, based on the size and mass of the particles, it is essential to ensure in addition that the radiomarker selectively/specifically labels the active substance without also labelling the carrier material.

The aim of the present invention is to prepare radioactively labelled microparticles which may be used in powdered formulations for use by inhalation and which meet the above requirement.

### DETAILED DESCRIPTION OF THE INVENTION

The invention relates to microparticles radioactively labelled with the technetium isotope ^{99m}Tc, the process for preparing such microparticles as well as the use of such microparticles for preparing a stable powder formulation. The microparticles according to the invention meet the requirements mentioned above.

Instead of the term “technetium isotope ^{99m}Tc” the abbreviation Tc* is also used hereinafter.

The formulations according to the invention do not differ in their pharmaceutical properties, such as e.g. their aerodynamic characteristics and dispersibility, from the formulations for pharmaceutical use in which the active substance is not labelled.

The radiolabelled microparticles prepared by the process according to the invention as well as the powder formulations for inhalation prepared from them have proved to be stable enough to allow so-called deposition studies to be carried out using them.

By microparticles are meant within the scope of the present invention pharmaceutical active substances in solid, preferably crystalline form, which have a mean particle size of about 0.5 to about 10 μm, preferably from about 1 to about 6 μm, particularly preferably from about 1.5 to about 5 μm. By the mean particle size in the sense used here is meant the 50% value from the distribution by volume, measured by laser diffraction using the dry dispersion method. Methods of determining the mean particle size are known from the prior art. For example, reference may be made to the remarks on this subject in the experimental section of WO 02/30389. Particles with the above-mentioned mean particle size are obtained for example by grinding (so-called micronising) crystals of a larger particle size, by special crystallisation processes or by spray drying processes. All these processes are known in the art.

Pharmaceutical active substances selected from among the anticholinergics, betamimetics, dopamine agonists, antiallergics, leukotriene antagonists and corticosteroids, and optionally combinations of active substances, are preferred within the scope of the present invention for preparing the radioactively labelled microparticles.

Examples of preferred anticholinergics are compounds selected from among the tiotropium salts, ipratropium salts, oxitropium salts,

salts of the compounds known from WO 02/32899
tropenol N-methyl-2,2-diphenylpropionate,
scopine N-methyl-2,2-diphenylpropionate,
scopine N-methyl-2-fluoro-2,2-diphenylacetate and
tropenol N-methyl-2-fluoro-2,2-diphenylacetate
   as well as salts of the compounds known from WO 02/32899
tropenol N-methyl-3,3′,4,4′-tetrafluorobenzilate,
scopine N-methyl-3,3′,4,4′-tetrafluorobenzilate;
scopine N-methyl-4,4′-dichlorobenzilate,
scopine N-methyl-4,4′-difluorobenzilate,
tropenol N-methyl-3,3′-difluorobenzilate,
scopine N-methyl-3,3′-difluorobenzilate and
tropenol N-ethyl-4,4′-difluorobenzilate, optionally in the form of their hydrates and solvates.

By salts are meant those compounds which contain, in addition to the abovementioned cations, as counter-ion, an anion with a single negative charge selected from among the chloride, bromide and methanesulphonate.

Particularly preferably the active substances within the scope of the present invention are the bromides or methanesulphonates of the abovementioned structures.

Of exceptional interest within the scope of the present invention are, for example, the anticholinergics tiotropium bromide, ipratropium bromide, oxitropium bromide, 2,2-diphenylpropionate tropenol-methobromide, scopine 2,2-diphenylpropionate-methobromide, scopine 2-fluoro-2,2-diphenylacetate-methobromide, tropenol 2-fluoro-2,2-diphenylacetate-methobromide, tropenol 3,3′,4,4′-tetrafluorobenzilate-methobromide, scopine 3,3′,4,4′-tetrafluorobenzilate-methobromide; scopine 4,4′-dichlorobenzilate-methobromide, scopine 4,4′-difluorobenzilate-methobromide, tropenol 3,3′-difluorobenzilate-methobromide, scopine 3,3′-difluorobenzilate-methobromide and tropenol 4,4′-difluorobenzilate-ethylbromide, while tiotropium bromide, ipratropium bromide, tropenol 2,2-diphenylpropionate-methobromide, scopine 2,2-diphenylpropionate-methobromide, scopine 2-fluoro-2,2-diphenylacetate-methobromide and tropenol 2-fluoro-2,2-diphenylacetate-methobromide are particularly important. Of outstanding importance is tiotropium bromide, particularly in the form of its crystalline monohydrate known from WO 02/30928.

Examples of betamimetics which may be used according to the invention are preferably compounds selected from among bambuterol, bitolterol, carbuterol, clenbuterol, fenoterol, formoterol, hexoprenaline, ibuterol, pirbuterol, procaterol, reproterol, salbutamol, salmeterol, sulphonterol, terbutaline, tulobuterol, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-on, 1-(4-amino-3-chloro-5-trifluoromethylphenyl)-2-tert.-butylamino)ethanol and 1-(4-ethoxycarbonylamino-3-cyano-5-fluorophenyl)-2-(tert.-butylamino)ethanol, optionally in the form of their racemates, their enantiomers, their diastereomers, as well as optionally their pharmacologically acceptable acid addition salts and hydrates. It is particularly preferable to use, as betamimetics, active substances of this kind selected from among fenoterol, formoterol, salmeterol, salbutamol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{(4-[3-(4-methoxyphenyl)-1,2,4-triazol-3-yl]-2-methyl-2-butylamino}ethanol, optionally in the form of their racemates, their enantiomers, their diastereomers, as well as optionally their pharmacologically acceptable acid addition salts and hydrates. Of the betamimetics mentioned above, the compounds formoterol and salmeterol, optionally in the form of their racemates, their enantiomers, their diastereomers, as well as optionally their pharmacologically acceptable acid addition salts and hydrates, are particularly important.

For example, the acid addition salts of the betamimetics selected from among the hydrochloride, hydrobromide, sulphate, phosphate, fumarate, methanesulphonate and xinafoate are preferred.

Particularly preferred in the case of formoterol are the salts selected from among the hydrochloride, sulphate and fumarate, of which the hydrochloride and fumarate are particularly preferred. According to the invention, formoterol fumarate is of exceptional importance.

Particularly preferred in the case of salmeterol are the salts selected from among the hydrochloride, sulphate and xinafoate, of which the xinafoate is particularly preferred.

Within the scope of the present invention, the corticosteroids which may be used according to the invention are compounds selected from among flunisolide, beclomethasone, triamcinolone, budesonide, fluticasone, mometasone, ciclesonide, rofleponide, GW 215864, KSR 592, ST-126 and dexamethasone. The preferred corticosteroids within the scope of the present invention are those selected from among flunisolide, beclomethasone, triamcinolone, budesonide, fluticasone, mometasone, ciclesonide and dexamethasone, while budesonide, fluticasone, mometasone and ciclesonide, especially budesonide and fluticasone, are of particular importance. The term steroids may be used on its own, within the scope of the present patent application, instead of the term corticosteroids. Any reference to steroids within the scope of the present invention also includes a reference to salts or derivatives which may be formed from the steroids. Examples of possible salts or derivatives include: sodium salts, sulphobenzoates, phosphates, isonicotinates, acetates, propionates, dihydrogen phosphates, palmitates, pivalates or furoates. The corticosteroids may optionally also be in the form of their hydrates.

Within the scope of the present invention, the term dopamine agonists denotes compounds selected from among bromocriptine, cabergolin, alpha-dihydroergocryptine, lisuride, pergolide, pramipexol, roxindol, ropinirol, talipexol, tergurid and viozan. It is preferable within the scope of the present invention to use dopamine agonists selected from among pramipexol, talipexol and viozan, pramipexol being of particular importance. Any reference to the abovementioned dopamine agonists also includes, within the scope of the present invention, a reference to any pharmacologically acceptable acid addition salts and hydrates thereof which may exist. By the physiologically acceptable acid addition salts thereof which may be formed by the abovementioned dopamine agonists are meant, for example, pharmaceutically acceptable salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid.

Examples of antiallergic agents which may be used according to the invention include epinastin, cetirizin, azelastin, fexofenadin, levocabastin, loratadine, mizolastin, ketotifen, emedastin, dimetinden, clemastine, bamipin, cexchloropheniramine, pheniramine, doxylamine, chlorophenoxamine, dimenhydrinate, diphenhydramine, promethazine, ebastin, desloratidine and meclizine. Preferred antiallergic agents which may be used within the scope of the present invention are selected from among epinastin, cetirizin, azelastin, fexofenadin, levocabastin, loratadine, ebastin, desloratidine and mizolastin, epinastin and desloratidine being particularly preferred. Any reference to the abovementioned antiallergic agents also includes, within the scope of the present invention, a reference to any pharmacologically acceptable acid addition salts thereof which may exist.

The microparticles according to the invention, radioactively labelled with the technetium isotope ^{99m}Tc (Tc*), may be prepared by a process which is characterised according to the invention in that a Tc* salt is taken up in a solvent, microparticles suspended in a suspension agent are added to this solution which is then mixed and finally the suspension agent and the solvent are eliminated.

Within the scope of the present invention the terms solvent and suspension agent refer to the dissolving properties with regard to the active substance which is to be labelled.

The radioactively labelled microparticles according to the invention may be prepared as follows, for example.

In order to radiolabel the microparticles, first the radioactive Tc* is obtained from an aqueous solution by known methods. For example, the technetium may be eluted in the form of pertechnetate ions (TcO₄⁻) from a Tc generator with NaCl solution and prepared after evaporating the aqueous solution to dryness. The pertechnetate salt NaTc*O₄ is present in admixture with NaCl.

The system for labelling a micronised powdered preparation as described herein is based on the procedure of dissolving radioactive Tc*, optionally mixing it with an (organic) suspension agent (based on the active substance which is to be labelled, as defined hereinbefore) and suspending the micronised active substance in this solution or mixture of solvent/suspension agent. To obtain the labelled particles, the solvent or the mixture of solvent/suspension agent is eliminated by evaporation, for example, so that the Tc* salt is deposited on the surface of the microparticles.

By radioactively labelled microparticles for the purposes of the present invention are meant those microparticles on whose surface radioactive Tc* is deposited, particularly in the form of a Tc* salt.

By Tc* salt for the purposes of the present invention are meant salts of the technetium isotope ^{99m}Tc, which are preferably selected from the group consisting of NaTc*O₄, ^{99m}Tc-TPAC (tetraphenylarsomium pertechnetate) and ^{99m}Tc-DTPA (diethylenetriaminepentaacetic acid), optionally mixed with NaCl.

Surprisingly it has been found that a uniform suspension of the micronised preparation in the suspension agent can be obtained by the addition of a small amount of a solvent in which the active substance which is to be suspended is theoretically soluble and that after evaporation of the mixture of solvent and suspension agent microparticles are formed which do not differ in their crystalline properties from the initial micronised preparation. Preferably, the above-mentioned solvent is selected so that it is also capable of dissolving the Tc* salt which is to be used.

The Tc* salt evaporated to dryness, particularly preferably in the form of the mixture of the salts NaTc*O₄/NaCl, is taken up in a solvent which is characterised by good dissolving properties for the Tc* salt. Alcohols, ethers, ketones, halohydrocarbons, polar organic solvents or mixtures of the abovementioned solvents have proved particularly suitable. If an alcohol is used this is preferably selected from among methanol, ethanol, propanol, butanol, glycol and isopropanol, ethanol being particularly preferred. If an ether is used this is preferably selected from among diethyl ether, methyltert-butyl-ether, tetrahydrofuran, dioxane and diisopropylether. If a ketone is used this is preferably selected from among acetone, methylethylketone and diethylketone. If a halohydrocarbon is used this is preferably selected from among dichloromethane or chloroform, of which dichloromethane is preferred. If a polar organic solvent is used this is preferably selected from among dimethylformamide, dimethylsulphoxide and acetonitrile. It is particularly preferable to use an alcohol, particularly ethanol, as solvent.

In the next step of the process a suspension agent is added to the solution of the Tc* salt in the solvent, this suspension agent preferably being selected so that, depending on the absolute quantity of salts present in the solution, the Tc* salt present is dissolved completely.

Suitable suspension agents according to the invention are selected from among the nonpolar, aprotic solvents consisting of the paraffins and olefins, preferably cyclohexane, n-heptane, pentane and n-hexane, preferably n-hexane. Particularly preferably, an amount of suspension agent is added such that the ratio of solvent to suspension agent is in the range between 1:50 and 1:1000, preferably in the range between about 1:100 and 1:500. The above values refer to the respective amounts by volume of the two components.

First, the liquid phase may be prepared with the Tc* salt dissolved in one of the above-mentioned solvents, to which the microparticles which are to be radiolabelled are then added.

Alternatively, a liquid phase may be prepared in which the Tc* salt is dissolved and which also contains the suspension agent in a low concentration, by first taking up the Tc* salt in the solvent and then adding a partial amount of the suspension agent to this solution. Then this solution is mixed with the micronised preparation which has previously been suspended in the remaining suspension agent. The mixture of solvent and suspension agent is then removed by known methods (e.g. by evaporation under reduced pressure). The radioactively labelled microparticles according to the invention are left.

The desired powder formulations for administration by inhalation are then obtained from these microparticles by methods known from the prior art.

These powder formulations contain the radioactively labelled microparticles according to the invention mixed with one or more suitable physiologically acceptable excipients.

The following physiologically acceptable excipients may be used according to the invention: monosaccharides (e.g. glucose or arabinose), disaccharides (e.g. lactose, saccharose, maltose), oligo- and polysaccharides (e.g. dextrans, starch, starch derivatives, cellulose, cellulose derivatives), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 500 μm, preferably between 10 and 250 μm, most preferably between 15 and 100 μm. In some cases it may be appropriate to add finer excipient fractions with an average particle size of about 1 to 9 μm to the excipients mentioned above. These finer excipients are also selected from the group of possible excipients listed hereinbefore.

In order to prepare the powder formulations, the radioactively labelled microparticles according to the invention are added to the excipient, optionally to the excipient mixture. Methods of preparing inhalable powders by grinding and micronising and by finally mixing the constituents are known from the prior art. For the preparation of the radioactively labelled inhalable powders or powder mixtures according to the invention in which a tiotropium salt is used as the pharmaceutical active substance, reference is made particularly to WO 02/30389, especially the remarks on page 5, line 29, to page 6, line 39, and the embodiments listed in Examples 1 to 4.

The inhalable powders according to the invention may be administered using powder inhalers known from the prior art. On this subject reference is made for example to the inhalers according to U.S. Pat. No. 4,570,630, U.S. Pat. No. 4,524,769, U.S. Pat. No. 4,627,432, U.S. Pat. No. 5,590,645, DE 36 25 685 or WO 94/28958.

The following detailed experimental descriptions serve to illustrate the invention more fully without restricting the scope of the invention to the embodiments by way of example described below.

Example of the Procedure for Preparing the Microparticles which May be Obtained by the Above Process (with Tiotropium Bromide as Active Substance)

The following steps describe the process for preparing radiolabelled microparticles:

1. Several ml of an aqueous sodium pertechnetate solution of the highest possible concentration (=V₁) with a total radioactivity of preferably 4,500-5,000 MBq are evaporated to dryness on a hotplate.
2. The residue is taken up in ethanol (=V₂) and evaporated down to a volume of ≧V3 on a hotplate.
3. The supernatant of the solution over the undissolved parts is quantitatively separated off (=V₃).
4. Micronised crystalline tiotropium bromide monohydrate (=m₁) and the suspension agent n-hexane (=V₄) are placed in a flask.
5. The ethanolic sodium pertechnetate solution (=V₃) is added thereto.
6. All the liquid constituents are distilled off to dryness from the resulting mixture, for example at 43° C. and under a reduced pressure of about 200 mbar.

The residue left in the flask corresponds to the microparticles which have been radiolabelled according to the invention and can be processed further to prepare a radioactively labelled powdered inhalant. Methods of doing this are known from the prior art (e.g. WO 02/30390).

Preferably, in step 2 of the process, the solution may be evaporated down to such an extent that only a smaller amount (e.g. 250 μl) can be taken up in step 3 and then by repeating steps 2 and 3 the total volume V₃ of the Tc*-ethanol solution is prepared as described above. In another alternative embodiment of the process the total volume of the Tc* solution can be increased by adding (e.g. 10 ml—partial amount of the solution V₄) pure suspension agent to the solution V₃ taken up, in step 3 of the process.

Similarly, the following procedure has proved particularly advantageous: the suspension according to step 4 of the process is prepared using only some of the suspension agent V₄ (e.g. 45 ml). To this is added half the Tc* solution according to the preferred process described above. Once this suspension has been evaporated down, the remaining quantity of n-hexane (e.g. 45 ml) is added to the suspension and the remaining Tc* solution is mixed with this suspension. This suspension is then further processed according to step 6 described above.

TABLE 1
Details of amounts and volumes

| | |
|---|---|
| Example 1 | Example 2 |

| | | | | |
|---|---|---|---|---|
| V₁ | 6 | ml | 1.5-2 | ml |
| V₂ | 10 | ml | 2 × 1 | ml |
| V₃ | 0.5 | ml | 2 × 250 | μl |
| V₄ | 99.5 | ml | 100 | ml |
| m₁ | 2 | g | 80 | mg |

1) A process for preparing microparticles radioactively labelled with the technetium isotope ^{99m}Tc (Tc*), wherein a Tc* salt is taken up in a solvent, the resulting solution is combined with microparticles suspended in a suspension agent and mixed, and the suspension agent and the solvent are removed.

2) The process according to claim 1 wherein the microparticles are solid pharmaceutical active substances which have a mean particle size of about 0.5 to about 10 μm.

3) The process according to claim 1 wherein the Tc* salt is selected from the group consisting of NaTc*O₄, Tc*-TPAC (tetraphenylarsomium pertechnetate) or Tc*-DTPA (diethylenetriaminepentaacetic acid), each optionally mixed with NaCl.

4) The process according to claim 1 wherein the solvent is selected from the group consisting of alcohols, ethers, ketones, halohydrocarbons, polar organic solvents and mixtures of the abovementioned solvents.

5) The process according to claim 1 wherein the suspension agent comprises a nonpolar aprotic solvent.

6) The process according to claim 1 wherein the ratio of solvent to suspension agent is in a range between 1:50 and 1:1000.

7) The process according to claim 1 wherein the radioactively labelled microparticles are prepared with a pharmaceutically active substance selected from the group consisting of anticholinergics, betamimetics, dopamine agonists, antiallergics, leukotriene antagonists and corticosteroids, and optionally combinations thereof.

8) The process according to claim 7 wherein the pharmaceutically active substance comprises anticholinergics selected from the group consisting of tiotropium salts, ipratropium salts, oxitropium salts, and salts of
tropenol N-methyl-2,2-diphenylpropionate,
scopine N-methyl-2,2-diphenylpropionate,
scopine N-methyl-2-fluoro-2,2-diphenylacetate and
tropenol N-methyl-2-fluoro-2,2-diphenylacetate
tropenol N-methyl-3,3′,4,4′-tetrafluorobenzilate,
scopine N-methyl-3,3′,4,4′-tetrafluorobenzilate;
scopine N-methyl-4,4′-dichlorobenzilate,
scopine N-methyl-4,4′-difluorobenzilate,
tropenol N-methyl-3,3′-difluorobenzilate,
scopine N-methyl-3,3′-difluorobenzilate, and
tropenol N-ethyl-4,4′-difluorobenzilate,
each optionally in the form of their hydrates or solvates.

9) The process according to claim 7 wherein the pharmaceutically active substance comprises betamimetics selected from among the group consisting of bambuterol, bitolterol, carbuterol, clenbuterol, fenoterol, formoterol, hexoprenaline, ibuterol, pirbuterol, procaterol, reproterol, salbutamol, salmeterol, sulphonterol, terbutaline, tolubuterol, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-on, 1-(4-amino-3-chloro-5-trifluoromethylphenyl)-2-tert.-butylamino)ethanol and 1-(4-ethoxycarbonylamino-3-cyano-5-fluorophenyl)-2-(tert.-butylamino)ethanol, each optionally in the form of their racemates, their enantiomers, and their diastereomers, and optionally their pharmacologically acceptable acid addition salts and hydrates.

10) The process according to claim 7 wherein the pharmaceutically active substance comprises corticosteroids selected from the group consisting of flunisolide, beclomethasone, triamcinolone, budesonide, fluticasone, mometasone, ciclesonide, rofleponide, GW 215864, KSR 592, ST-126 and dexamethasone, and combinations thereof.

11) The process according to claim 7 wherein the pharmaceutically active substance comprises dopamine agonists selected from the group consisting of bromocriptine, cabergolin, alpha-dihydroergocryptine, lisuride, pergolide, pramipexol, roxindol, ropinirol, talipexol, tergurid and Viozan, and combinations thereof.

12) The process according to claim 7 wherein the pharmaceutically active substance comprises antiallergics selected from the group consisting of epinastin, cetirizin, azelastin, fexofenadin, levocabastin, loratadine, mizolastin, ketotifen, emedastin, dimetinden, clemastine, bamipin, cexchloropheniramine, pheniramine, doxylamine, chlorophenoxamine, dimenhydrinate, diphenhydramine, promethazine, ebastin, desloratidine and meclizine, and combinations thereof.

13) Microparticles radioactively labelled with the technetium isotope (Tc*) prepared by the process according to claim 1.

14) A method for administering the microparticles according to claim 13 to a subject comprising preparing a powder comprising the microparticles and administering the powder to the subject by inhalation.
